(19) United States
(12) Patent Application Publication
(10) Pub. No.: US 2026/0242431 A1
(43) Pub. Date: Aug. 20, 2026

(54) IMPROVED LEGUME PROTEINS

(71) Applicant: ROQUETTE FRERES, Lestrem (FR)

(72) Inventors: Guillaume FARDIN, LESTREM (FR); Aline LECOCQ, MOUVAUX (FR)

(21) Appl. No.: 19/155,133

(22) PCT Filed: Feb. 9, 2024
(86) PCT No.: PCT/EP2024/025074

§ 371 Date: Aug. 8, 2025

(30) Foreign Application Priority Data
Feb. 10, 2023 (FR) 2301275

(51) Int. Cl. C07K 14/415; A23C 11/10; A23J 1/14; A23L 11/60; A23L 29/238; B01D 61/14; B01D 69/02; C07K 1/34

(52) U.S. Cl. CPC C07K 14/415; A23C 11/103; A23J 1/14; A23L 11/60; A23L 29/238; B01D 61/145; B01D 69/02; C07K 1/34; B01D 2315/16; B01D 2325/20

(57) ABSTRACT

Legume proteins, preferentially pea or faba bean proteins, having a milky aromatic profile, to a method for producing said legume proteins, preferentially pea or faba bean proteins, and to the use of said proteins for producing food or beverage products, in particular plant-based alternatives to milk.

### FIELD OF THE INVENTION

The subject matter of the invention is a composition comprising new legume proteins, preferentially pea or faba bean proteins, characterized by an innovative nutritional and functional profile. Another subject matter of the invention relates to a method for producing these new legume proteins, preferentially pea or faba bean proteins. The invention also relates to the use of said legume proteins, preferentially pea or faba bean proteins, in the production of food products.

### PRIOR ART

Daily requirements for proteins are generally between 12 and 20% of food intake. These proteins are provided equally by products of animal origin (meat, fish, eggs, dairy products) and by plant-based food (cereals, leguminous plants, seaweed).

In developed countries, protein intake is still today predominantly in the form of proteins of animal origin. These proteins have good nutritional properties and interesting functional properties, which allow them to be used in a very wide variety of food products.

However, numerous studies show that excessive consumption of proteins of animal origin to the detriment of plant proteins is one of the causes of increases in cancer and cardiovascular diseases. Moreover, animal proteins have many drawbacks, both in terms of their allergenicity (especially proteins from milk or eggs) and in environmental terms, in connection with the harmful effects of intensive farming.

Thus, there is an increasing demand from manufacturers for proteins of plant origin having beneficial nutritional and functional properties without, however, having the disadvantages of proteins of animal origin.

Since the 1970s, the pea is the pulse plant which has been the most developed in Europe, predominantly in France, especially as a protein resource intended for animal and human food.

First of all, there are the so-called “dry extraction” methods for extracting legume protein, preferentially pea or faba bean protein. The principle of these methods is to mill the seed into flour, which is then introduced into a turbo separator, a device used to classify particles according to size and density within an air stream. Turbo separation produces a protein-enriched fraction and a starch-enriched fraction. As will be explained later, the protein-enriched fraction is about 40%-60% and still contains between 2% and 15% starch. This starch is a polysaccharide fraction that is not necessarily desired, as it contributes to raising blood sugar levels; replacing it with polysaccharides that are non-digestible by consumers but digestible by their digestive microflora would be of interest.

Mention may be made, as an example of a method for extracting pea protein, of patent EP1400537, which is typical of so-called “wet” extraction methods. In this method, the seed is milled in the absence of water (method referred to as “dry milling”) in order to obtain a flour. This flour is then suspended in water at room temperature to then proceed with the various steps for extracting the protein. This type of method separates proteins belonging to the globulin subgroup (about 80% of pea proteins) and proteins belonging to the albumin subgroup (about 20% of pea proteins) by isoelectric precipitation. These remain in the liquid fraction after recovery of the floc, which is mainly composed of globulins. These albumins are in solution with pea galactooligosaccharides (GOS), which are not easily digested by humans, salts, mainly potassium, which can be harmful in high doses, and other anti-nutritional factors such as anti-trypsin factors.

Several problems are easy to guess, such as proteins being separated into two fractions, requiring that value be added to two different fractions, or GOS being combined with albumins, which are difficult to digest and require post-treatment, making the method more complex and costly. Lastly, the massive presence of starch in the protein-enriched fraction of concentrates obtained by dry turbo separation is also a nutritional disadvantage for certain formulations.

Another wet method alternative consists in replacing the isoelectric precipitation step with a membrane filtration step. This membrane separation concentrates the globulin and albumin protein fractions in the retentate, while eliminating some of the salts and sugars in the permeate. The article “Impact of processing on functional properties of protein products from wrinkled peas” by Fuhrmeister & al. (Journal of Food Engineering, Volume 56, Issues 2-3, February 2003, Pages 119-129) describes such a method and compares it to isoelectric precipitation. The article concludes that the 50 KDa cut-off is the one to consider in order to optimize filtration throughput and recovery yield (but at the cost of a more unbalanced retentate protein composition).

Before being fed to the membrane filtration system, the pea seed conventionally undergoes two types of treatment at the start of the method: either the seed is milled as in patent application U.S. Pat. No. 10,143,226 or WO2018226689, or it undergoes a soaking step as described in the Fuhrmeister article cited above.

After a great deal of research, the applicant has developed a new manufacturing method for supplying legume proteins, preferentially pea or faba bean proteins, which contain all the so-called soluble legume proteins, preferentially pea proteins, that is, comprising all the globulins and albumins, by combining a wet blanching step and an ultrafiltration step with a cut-off selected between 5 KDa and 10 KDa. Said legume proteins, preferentially pea or faba bean proteins, also preserve their functional properties, in particular their gelling and emulsifying properties, as well as their organoleptic profile.

### SUMMARY OF THE INVENTION

Thus, the invention relates to a method for producing legume protein, preferentially pea or faba bean protein, comprising the following steps:
1. Preparation of an aqueous suspension of legume seeds, preferentially pea or faba bean seeds, milled in an aqueous solution, said preparation being carried out in the presence of a heat treatment;
2. Removal of an insoluble fraction by solid/liquid separation of the aqueous suspension of milled legume seeds, preferentially pea or faba bean seeds, obtained in step 1) to obtain a protein-enriched fraction;
3. Ultrafiltration of the protein-enriched fraction and removal of a permeate to generate a retentate

In a preferred embodiment, the invention relates to a method for producing legume protein, preferentially pea or faba bean protein, consisting of the following sequence of steps:
1. Preparation of an aqueous suspension of legume seeds, preferentially pea or faba bean seeds, milled in an aqueous solution, said preparation being carried out in the presence of a heat treatment;
2. Removal of an insoluble fraction by solid/liquid separation of the aqueous suspension of milled legume seeds, preferentially pea or faba bean seeds, obtained in step 1) to obtain a protein-enriched fraction;
3. Ultrafiltration of the protein-enriched fraction and removal of a permeate to generate a retentate.

Preferentially, the leguminous plants, preferentially peas, are introduced in the form of whole legume seeds, preferentially pea or faba bean seeds, into the aqueous solution and step 1 of the method comprises a step of wet milling the aqueous composition formed between the legume seeds, preferentially pea or faba bean seeds, and the aqueous solution so as to obtain the aqueous suspension of milled legume seeds, preferentially pea or faba bean seeds.

Preferably, the heat treatment in step 1 comprises:
a) introducing legume seeds, preferentially pea or faba bean seeds, or milled legume seeds, preferentially pea or faba bean seeds, into an aqueous solution at a temperature of between 65° C. and 90° C. so as to obtain an aqueous composition;
b) heat treating the aqueous composition obtained in step a) formed between the legume seeds, preferentially pea or faba bean seeds, or the milled legume seeds, preferentially pea or faba bean seeds, and the aqueous solution at a temperature of between 40° C. and 65° C. for 1 to 10 min.

Preferably, the ultrafiltration in step 3 is carried out using a membrane with a cut-off value of between 5 KDa and 10 KDa.

Another subject matter of the invention relates to the legume protein, preferentially pea or faba bean protein, that can be obtained by the method of the invention.

Preferably, the legume protein composition, preferentially pea or faba bean, that can be obtained by the method of the invention is characterized in that it comprises a protein content of between 70% and 90% expressed in grams of protein per 100 g of dry matter, said proteins consisting of a mixture of globulins and albumins.

Preferably, the dry weight ratio of globulins to albumins is between 70/30 and 90/10, preferentially between 75/25 and 85/15.

Another subject matter of the invention also relates to the use of said legume protein, preferentially pea or faba bean protein, for producing food or beverage products, especially plant-based alternatives to milk.

### DETAILED DESCRIPTION OF THE INVENTION

The invention relates to a method for producing legume protein, preferentially pea or faba bean protein, comprising the following steps:
1. Preparation of an aqueous suspension of legume seeds, preferentially pea or faba bean seeds, milled in an aqueous solution, said preparation being carried out in the presence of a heat treatment;
2. Removal of an insoluble fraction by solid liquid separation of the aqueous suspension of milled legume seeds, preferentially pea or faba bean seeds, prepared in step 1) to obtain a protein-enriched fraction;
3. Ultrafiltration of the protein-enriched fraction and removal of a permeate to generate a retentate.

### Step 1.

Step 1) comprises introducing a legume, preferentially pea or faba bean, into an aqueous solution. The legume seeds, preferentially pea or faba bean seeds, used in step 1) may have been previously subjected to steps that are well known to those skilled in the art, such as especially cleaning (removal of undesired particles such as stones, dead insects, soil residues, etc.) or even the removal of the external fibers of the peas (external cellulose hull) through a well-known step referred to as “dehulling”.

Thus, “legumes” in step 1) refers to complete legumes or legume cotyledons, from which the external hull has preferentially been removed. Alternatively, milled legume seeds (that is, legume flour) can be used, these milled legumes generally being obtained by dry milling.

The legume protein according to the invention is selected from soybean, beans, peas, faba beans, chickpeas, peanuts, cultivated lentils, cultivated alfalfa, various clovers, broad beans, carob and licorice. Preferably, legume protein according to the invention is selected from pea and faba bean. Even more preferably, legume protein according to the invention is pea.

The pea contains approximately 27% by weight of protein substances. The term “pea” is considered here in its broadest accepted use and includes, in particular, all the wild varieties of “smooth pea” and all the mutant varieties of “smooth pea” and “wrinkled pea”, regardless of the uses for which said varieties are usually intended (human food, animal feed and/or other uses). Pea protein, predominantly pea globulin, has been extracted and utilized industrially for a great number of years.

Thus, “peas” in step 1) refers to complete peas or pea cotyledons, from which the external hull has preferentially been removed. Alternatively, milled pea seeds (that is, pea flour) can be used, these milled peas generally being obtained by dry milling.

The aqueous solution may be water, and may also comprise additives such as anti-foaming or bacteriostatic compounds.

Especially, the ratio by weight of amount of legumes to amount of aqueous solution in step 1) can be between 0.5 and 2. The ratio can therefore be 0.5; 0.6; 0.7; 0.8; 0.9; 1.0; 1.1; 1.2; 1.3; 1.4; 1.5; 1.6; 1.7; 1.8; 1.9; 2.0, as well as all the ranges obtained with these values as upper and lower limits.

Preferably, the ratio by weight of amount of peas to amount of aqueous solution in step 1) can be between 0.5 and 2. The ratio can therefore be 0.5; 0.6; 0.7; 0.8; 0.9; 1.0; 1.1; 1.2; 1.3; 1.4; 1.5; 1.6; 1.7; 1.8; 1.9; 2.0, as well as all the ranges obtained with these values as upper and lower limits.

Alternatively, the pH of the suspension of step 1) is adjusted to between 8 and 10. The pH can therefore be 8.0; 8.1; 8.2; 8.3; 8.4; 8.5; 8.6; 8.7; 8.8; 8.9; 9.0; 9.1; 9.2; 9.3; 9.4; 9.5; 9.6; 9.7; 9.8; 9.9; 10.0, as well as all the ranges obtained with these values as upper and lower limits.

This adjustment can be made by adding a base such as sodium hydroxide, lime or potash, preferentially sodium hydroxide. According to another variant, the pH is not adjusted in this step.

According to a preferred embodiment, the temperature of the aqueous solution is preheated between 65° C. and 90° C. Heating can be carried out using any installation well known to those skilled in the art, such as an immersed heat exchanger. Preferentially, the temperature is between 70° C. and 80° C. or even about 75° C. The preheating temperature, expressed in ° C., can therefore be 65, 66, 67, 68, 69, 70, 71, 72, 73, 74, 75, 76, 77, 78, 79, 80, 81, 82, 83, 84, 85, 86, 87, 88, 89, 90, as well as all the ranges obtained with these values as upper and lower limits.

The aqueous suspension of milled legume seeds, preferentially pea or faba bean seeds, is obtained in step 1) by introducing the milled legume(s) into the aqueous solution, which may optionally have been pre-heated if pre-treatment is implemented.

Preferably, the heat treatment of the aqueous composition obtained in step 1 is carried out at a temperature of between 40° C. and 65° C. for 1 to 10 minutes. The temperature, expressed in ° C., can therefore be 40, 41, 42, 43, 44, 45, 46, 47, 48, 49, 50, 51, 52, 53, 54, 55, 56, 57, 58, 59, 60, 61, 62, 63, 64, 65 and all the ranges obtained with these values as upper and lower limits. The heating time, expressed in minutes, can therefore be 1, 2, 3, 4, 5, 6, 7, 8, 9, 10, and all the ranges obtained with these values as upper and lower limits.

The aqueous composition can be heated or cooled to reach this temperature. Preferably, the heat treatment temperature is between 40° C. and 60° C., or even between 45° C. and 55° C. Preferentially, the heat treatment is carried out for 2 to 4 min.

In the case where legume seeds, preferentially pea or faba bean seeds (and not already milled legume flour, preferentially pea or faba bean flour), are used in the preparation of the suspension according to step 1), the method comprises a step of wet milling the aqueous composition obtained between the legume seeds, preferentially pea or faba bean seeds, and the aqueous solution to obtain an aqueous suspension of milled legume seeds, preferentially pea or faba bean seeds. Wet milling takes place after said heat treatment. Preferably, the method is carried out using legume seeds, preferentially pea or faba bean seeds, and the wet milling step is carried out by continuous passage through one or more mills in order to obtain the aqueous suspension of leguminous plants, preferentially of pea or faba bean milled seeds. The one or more mills can be any type of mill suitable for wet milling, such as wet ball mills, wet conical mills, wet helical mills or wet mills equipped with rotor-stator systems. According to one variant, the mill can be the one used in the examples of document WO2019/053387 in the name of the Applicant. In the variant where the mill is of the rotor-stator type, this type of mill can enable continuous milling by passing the aqueous composition obtained between the legume seeds, preferentially pea or faba bean seeds, and the aqueous solution in said mill. According to one preferred sub-variant, the method combines two cutting stages (pre-cutting then cutting) using different rotor-stator mills for each one of these cuts. The pre-cutting and cutting can be carried out one after the other or, alternatively, the cutting can take place after pre-cutting and storing the aqueous composition obtained between the legume seeds, preferentially pea or faba bean seeds, and the treated aqueous solution. Such mills are disclosed in document WO2019/158589. Optionally, a dilution with water can be carried out during or at the end of this step in order to form the aqueous suspension of milled legume seeds, preferentially pea or faba bean seeds. Alternatively, during milling, water is added continuously or discontinuously to dilute the mixture. Generally, the solids content of the aqueous suspension of milled legume seeds, preferentially pea or faba bean seeds, ranges from 10% to 30%, for example from 15% to 25%.

As will be shown in the section of the description dedicated to examples, the synergy of a heat treatment, preferentially carried out at a temperature of between 40° C. and 65° C. for 1 to 10 minutes, even more preferentially coupled with wet milling in step 1, and ultrafiltration in step 3, makes it possible to obtain a legume protein, preferentially a pea or faba bean protein, with both high gelling power and an organoleptic profile compatible with a maximum number of industrial applications, including food applications.

Step 2) of the method consists in extracting components from the aqueous suspension of milled legume seeds, preferentially pea or faba bean seeds, and in particular in extracting a protein-enriched fraction by solid-liquid separation from the aqueous suspension of milled legume seeds, preferentially pea or faba bean seeds. Alternatively, before carrying out the solid-liquid separation stage, it is possible to carry out a stage of adjusting the pH of the aqueous suspension of milled legume seeds, preferentially pea or faba bean seeds. The solid-liquid separation can thus take place after adjusting the aqueous suspension of legume seeds, preferentially pea or faba bean seeds, to a pH ranging from 6 to 9, preferably from 8 to 9, most preferentially from 8.5 to 9. The pH can therefore be 6.0; 6.5; 7.0; 7.5; 8.0; 8.5; 9.0, as well as all the ranges obtained with these values as upper and lower limits.

This pH adjustment stage can be carried out in a stirred tank. This stage can be shorter or longer, and last from 1 to 240 minutes, for example, generally from 5 to 60 minutes. The pH adjustment time, expressed in minutes, can therefore be 1, 5, 10, 15, 20, 25, 3á, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, as well as all the ranges obtained with these values as upper and lower limits.

To perform the pH adjustment, any type of acid and/or base, organic or inorganic, or mixtures thereof, can be added. Examples of acids that can be used include hydrochloric acid, sulfuric acid, citric acid or mixtures thereof. As an example of a base, mention may be made of sodium hydroxide, potash or lime and the mixtures thereof. This addition of base or acid and the pH measurement can be carried out online. The base and/or acid may be in the form of aqueous solutions. Advantageously, prior to this solid-liquid separation, the aqueous suspension of milled legume seeds, preferentially pea or faba bean seeds, is cooled to a temperature below 15° C. This temperature can in particular range from 4 to 14° C., for example 10 to 12° C. The cooling temperature expressed in ° C. can therefore be 0, 1, 2, 3, 4, 5, 6, 7, 8, 9, 10, 11, 12, 13, 14, 15, as well as all the ranges obtained with these values as upper and lower limits.

This cooling step can be carried out using known techniques, such as passing the aqueous suspension of milled legume seeds, preferentially pea or faba bean seeds, through a heat exchanger.

Generally, the protein-enriched fraction is the soluble part of the aqueous suspension of legume seeds, preferentially pea or faba bean seeds, and the starch- and fiber-rich fraction is the insoluble part. It is also possible to separate more than two insoluble fractions and, for example, to recover a first insoluble fraction that is richer in starch and a second insoluble fraction that is richer in fiber. Thus, according to one variant of the method, a starch-rich fraction and/or a fiber-rich fraction is recovered from the insoluble part resulting from the removal step 2).

“Starch-rich fraction and fiber-rich fraction”, or “insoluble fraction”, generally refers to a fraction comprising at least 50% of starch and/or fiber. The methods for quantifying starch and fiber are known to those skilled in the art, and specific methods are indicated later in the description. These fractions are conventionally recovered by the known separation methods. The solid-liquid separation may especially be carried out by means of at least one separation step with a decanter, especially a centrifugal decanter, a centrifuge or else with hydrocyclones. The method can likewise make it possible to recover one or more fiber- and/or starch-enriched fractions that are removed from the suspension, and to recover the protein-enriched fraction that is useful for the rest of the method of the invention.

### Step 3)

The method then includes a step of ultrafiltration of the protein-enriched fraction and removal of a permeate to generate a protein-enriched retentate.

“Ultrafiltration” is a membrane separation method distinguished from microfiltration or nanofiltration by the size of the particles in suspension or solution that can pass through it. For ultrafiltration, this size is between 1 and 100 nanometers (nm).

Preferably, ultrafiltration is performed with a cut-off selected between 5 KDa and 10 KDa (KDa standing for Kilodaltons). This selection runs counter to earlier work by Fuhrmeister & al., who advocate the use of membranes with a 50 KDa cut-off as the best choice between yield and productivity. Selecting a cut-off between 5 KDa and 10 KDa allows more albumin to be retained. The cut-off points can therefore be 5 KDa, 6 KDa, 7 KDa, 8 KDa, 9 KDa and 10 KDa, as well as all the ranges formed by these values.

Preferably, the filtration temperature is between 45° C. and 60° C. The filtration temperature can therefore be 45° C., 46° C., 47° C., 48° C., 49° C., 50° C., 51° C., 52° C., 53° C., 54° C., 55° C., 56° C., 57° C., 58° C., 59° C. or 60° C., as well as all the ranges formed by these values.

Preferably, the transmembrane pressure will be between 1 and 5 bar, preferentially between 2 and 4 bar. Transmembrane pressure values can be 1 bar, 2 bar, 3 bar or 4 bar, as well as all the ranges formed by these values. Transmembrane pressure is a parameter well known to those skilled in the art, and consists of the pressure difference on either side of the ultrafiltration membrane.

Ultrafiltration is preferably carried out in a tangential filtration module.

Even more preferably, ultrafiltration is followed by a diafiltration step. Diafiltration consists of successive stages of concentration of the ultrafiltration retentate and addition of water to increase the purity of the retentate. Preferably, diafiltration is carried out to achieve a minimum protein content of 70%. The protein content will therefore be at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, as well as all the ranges formed by these values.

Advantageously, when ultrafiltration is used in the method in combination with the heat treatment described above in step 1, as will be demonstrated in the example section, a legume protein, preferentially pea or faba bean protein, is obtained, with a minimum protein content of 70% and with preserved gelling and emulsifying properties and organoleptic profile.

Method steps 1), 2) and 3) are carried out in this order. However, other optional steps can be implemented between steps 1), 2) and 3), such as pH adjustment, if this is deemed necessary by the person skilled in the art.

At the end of step 3), the method may comprise a step 4) of adjusting the pH of the legume protein, preferentially pea or faba bean protein, to a pH ranging from 6 to 7.5, generally from 6.5 to 7.5. This step can be carried out by adding an inorganic or organic base, such as by adding sodium hydroxide. The pH is generally raised by adding a basic aqueous solution.

Preferably, the method can then comprise a step 5) of additional heat treatment of the legume protein, preferentially pea or faba bean protein. The temperature and time conditions can vary widely in this step, for example from 70° C. to 140° C. and lasting from 0.1 seconds to several minutes. According to a first variant of this step of additional heat treatment, the temperature ranges from 70° C. to 90° C. and its duration ranges from 0.1 seconds to 30 minutes. According to a second variant of this step of additional heat treatment, the temperature ranges from 90° C. to 110° C. and its duration ranges from 0.1 seconds to 5 minutes. According to another variant, this step of additional heat treatment is carried out at a temperature ranging from 110° C. to 140° C. for a time ranging from 0.1 to 30 seconds, preferentially from 0.2 to 15 seconds, for example from 0.3 to 10 seconds. The aim of this step may be to functionalize and/or sanitize the legume protein, preferentially pea or faba bean protein. To perform this step of additional heat treatment, the legume protein, preferentially pea or faba bean protein, can be in the form of an aqueous dispersion, preferentially having a solids content ranging from 10 to 25%, for example from 15 to 20%. Advantageously, the method of the invention comprises, following the additional heat treatment step, a step f1) of cooling of the legume protein, preferentially pea or faba bean protein. According to one preferred variant, this cooling step is obtained by flash-cooling. At the end of this step, the temperature can range from 60° C. to 100° C., for example between 70° C. and 90° C. Similarly, this flash-cooling step is carried out by applying a vacuum to the aqueous dispersion of legume protein, preferentially pea or faba bean protein, the vacuum applied being determined based on the chosen cooling temperature.

According to one variant of the method, it comprises a step 6a) of shearing the legume protein, preferentially pea or faba bean protein, for example by passing the aqueous dispersion of proteins through a high-pressure pump. As examples of high-pressure pumps, mention may be made of the high-pressure pumps marketed by Silverson, also referred to as “high shear mixers”, such as those in the UHS range. Preferably, the shearing step is performed by a high-pressure pump.

The shearing step can take place before or after the steps of heat treatment and/or raising the pH.

According to another variant, the method alternatively comprises a step 6b) of homogenizing the legume protein, preferentially pea or faba bean protein.

Any type of homogenizer can be used to perform this homogenization step. According to the invention, it refers to equipment comprising a high-pressure pump and a homogenizing head, wherein the equipment is designed so that the product to be homogenized passes under pressure through this homogenizing head. A homogenizing head consists of a reduced opening, generally comprising a seat, a valve and an impact ring. Passing the aqueous dispersion of legume protein, preferentially pea or faba bean protein, through the homogenizer can thus make it possible to homogenize the legume protein, preferentially pea or faba bean protein. The homogenization can be low-pressure homogenization, high-pressure homogenization or ultra-high-pressure homogenization. Depending on the homogenization technique used, the homogenization pressure can vary widely from 1 to 1000 bar, for example from 20 to 800 bar. According to one variant, the homogenization pressure ranges from 20 to 200 bar, for example from 50 to 150 bar. According to another variant, the homogenization pressure ranges from 200 to 800 bar, for example from 300 to 800 bar. According to one variant, the homogenization is a single-action homogenization. According to another variant, the homogenization is a multiple-action homogenization, for example a double-action homogenization. The homogenizers that can be used are marketed by GEA or Tetra Pak, for example.

The homogenization step can take place before or after the steps of heat treatment and/or raising the pH.

The method according to the invention can also comprise a step 7) of drying the legume protein, preferentially pea or faba bean protein. Generally, this drying step is carried out so as to reach a solids content greater than 80%, preferentially greater than 90%, most preferentially greater than 94% by weight of solids relative to the weight of the legume protein, preferentially pea or faba bean protein. To this end, any technique well known to those skilled in the art can be used, for instance freeze-drying, flash drying or drying on a drying cylinder, or atomization. The process may also comprise a step of milling or micronizing. Atomization is the preferred technology, in particular multiple-effect atomization. The legume protein, preferentially pea or faba bean protein, may be in the form of a powder having a particle size d50, which can vary widely, for example from 10 μm to 500 μm, generally from 50 μm to 150 μm.

In the present invention, the term “D50” means the particle size measured in microns separating two populations by number, containing respectively 50% and 50% of the total amount of particles of the protein composition.

For performing this d50 measurement, a laser particle size analyzer is preferentially used, even more preferentially the Mastersizer 2000 from Malvern. The parameters used are as follows: Liquid application, dispersed in ethyl alcohol; refractive index: 1.52; absorption index: 0.1; no ultrasound used.

Steps 1) to 7) of the method can be carried out in this precise order but, depending on the needs of the person skilled in the art, other optional steps can be implemented, such as pH adjustment.

In a preferred embodiment, the invention relates to a method for producing legume protein, preferentially pea or faba bean protein, consisting of the following sequence of steps 1, 2 and 3.

In this preferred embodiment, the invention relates to a method for producing legume protein, preferentially pea or faba bean protein, consisting of the following sequence of steps:
1. Preparation of an aqueous suspension of legume seeds, preferentially pea or faba bean seeds, milled in an aqueous solution, said preparation being carried out in the presence of a heat treatment;
2. Removal of an insoluble fraction by solid/liquid separation of the aqueous suspension of milled legume seeds, preferentially pea or faba bean seeds, obtained in step 1) to obtain a protein-enriched fraction;
3. Ultrafiltration of the protein-enriched fraction and removal of a permeate to generate a retentate

In another preferred embodiment, the invention relates to a method for producing legume protein, preferentially pea or faba bean protein, consisting of the following sequence of steps 1, 2, 3, 4, 5 and 7.

In this preferred embodiment, the invention therefore relates to a method for producing legume protein, preferentially pea or faba bean protein, consisting of the following sequence of steps:
1. Preparation of an aqueous suspension of legume seeds, preferentially pea or faba bean seeds, milled in an aqueous solution, said preparation being carried out in the presence of a heat treatment;
2. Removal of an insoluble fraction by solid/liquid separation of the aqueous suspension of milled legume seeds, preferentially pea or faba bean seeds, obtained in step 1) to obtain a protein-enriched fraction;
3. Ultrafiltration of the protein-enriched fraction and removal of a permeate to generate a retentate
4. Adjustment of the pH of the legume protein, preferentially pea or faba bean protein, to a pH ranging from 6 to 7.5, generally from 6.5 to 7.5.
5. Additional heat treatment of the legume protein, preferentially pea or faba bean protein. The temperature and time conditions can vary widely in this step, for example from 70° C. to 140° C. and lasting from 0.1 seconds to several minutes
6. Drying the legume protein, preferentially pea or faba bean protein, so as to reach a solids content greater than 80%, preferentially greater than 90%, most preferentially greater than 94% by weight of solids relative to the weight of said legume protein, preferentially pea or faba bean protein

In another preferred embodiment, the invention relates to a method for producing legume protein, preferentially pea or faba bean protein, consisting of the following sequence of steps 1, 2, 3, 4, 5, 6a and 7.

In another preferred embodiment, the invention relates to a method for producing legume protein, preferentially pea or faba bean protein, consisting of the following sequence of steps 1, 2, 3, 4, 5, 6b and 7.

In another preferred embodiment, the invention relates to a method for producing legume protein, preferentially pea or faba bean protein, consisting of the following sequence of steps 1, 2, 3 and 7.

In this preferred embodiment, the invention therefore relates to a method for producing legume protein, preferentially pea or faba bean protein, consisting of the following sequence of steps:
1. Preparation of an aqueous suspension of legume seeds, preferentially pea or faba bean seeds, milled in an aqueous solution, said preparation being carried out in the presence of a heat treatment;
2. Removal of an insoluble fraction by solid/liquid separation of the aqueous suspension of milled legume seeds, preferentially pea or faba bean seeds, obtained in step 1) to obtain a protein-enriched fraction;
3. Ultrafiltration of the protein-enriched fraction and removal of a permeate to generate a retentate
4. Drying the legume protein, preferentially pea or faba bean protein, so as to reach a solids content greater than 80%, preferentially greater than 90%, most preferentially greater than 94% by weight of solids relative to the weight of said legume protein, preferentially pea or faba bean protein

Another subject matter of the invention relates to a legume protein, preferentially pea or faba bean protein, that can be obtained by the method of the invention.

Another object of the invention relates to a legume protein composition, preferentially pea or faba bean protein composition, characterized in that it comprises a protein content of between 70% and 90% expressed in grams of protein per 100 g of dry matter, said proteins consisting of a mixture of globulins and albumins.

“Legume protein composition” means a composition that comprises mainly, but not exclusively, legume proteins. Such a composition may contain residual impurities such as, for example, minerals, sugars and so on.

The term “leguminous” is considered herein to mean the family of dicotyledonous plants of the order Fabales. This is one of the largest flowering plant families, third after Orchidaceae and Asteraceae in terms of number of species. It contains approximately 765 genera, bringing together more than 19,500 species. Several leguminous plants are important crop plants, including soybean, beans, peas, faba beans, chickpeas, peanuts, cultivated lentils, cultivated alfalfa, various clovers, broad beans, carob and licorice.

The term “pea” should be understood in the present application as all the wild varieties of “smooth pea” and all the mutant varieties of “smooth pea” and “wrinkled pea”. The term “pea” is considered here in its broadest accepted use and includes in particular all the varieties of “smooth pea” and “wrinkled pea” and all the mutant varieties of “smooth pea” and “wrinkled pea”, regardless of the uses for which said varieties are usually intended (human food, animal feed and/or other uses). The term “pea” in the present application includes pea varieties belonging to the Pisum genus and more particularly to the species sativum and aestivum. Said mutant varieties are in particular those named “r mutants”, “rb mutants”, “rug 3 mutants”, “rug 4 mutants”, “rug 5 mutants” and “lam mutants” as described in the article by C-L HEYDLEY et al., entitled “Developing novel pea starches” Proceedings of the Symposium of the Industrial Biochemistry and Biotechnology Group of the Biochemical Society, 1996, pages 77-87.

The term “protein” should be understood in the present application to mean the macromolecules formed from one or more polypeptide chains consisting of a sequence of amino acid residues bonded to one another by peptide bonds. In the particular context of pea proteins, the present invention relates more particularly to globulins (about 50-60% by weight of the pea proteins) and albumins (20-25% by weight of the pea proteins).

The term “globulin”, for the purposes of the present invention, means proteins that are soluble in neutral saline solutions. Legume globulins, preferentially pea or faba bean globulins, are mainly subdivided into three sub-families: legumins, vicilins and convicilins.

The term “albumin”, for the purposes of the present invention, means proteins that are soluble in pure water. Pea albumins, which are present in legume proteins, preferentially pea or faba bean proteins, at about 20%, are mainly subdivided into two families named PA1 and PA2. Legume albumins, preferentially pea or faba bean albumins, are mainly subdivided into two families named PA1 and PA2.

Generally, the protein content by weight of the legume protein, preferentially pea or faba bean protein, according to the invention is between 70% and 90% expressed in grams of protein per 100 g of dry matter. The protein content is the N6.25 content, calculated using the Dumas method. The protein content is between 71% and 90%, comprising 71%, 72%, 73%, 74%, 75%, 76%, 77%, 78%, 79%, 80%, 81%, 82%, 83%, 84%, 85%, 86%, 87%, 88%, 89 or 90%.

It obviously generally comprises other minority constituents other than proteins, such as starch, lipids, fibers, and/or sugars. Generally, the total starch content in the legume protein, preferentially pea or faba bean protein, produced according to the method of the invention ranges from 0% to 20%, for example from 0% to 1%, especially from 0.5% to 5%. This total starch content can be measured using the AOAC 996.11 method. Generally, the total lipid content is from 0% to 15%, for example from 1% to 10%. The total lipid content can be determined by the AOAC 996.06 acid hydrolysis method.

Preferably, the dry weight ratio of globulins to albumins is between 70/30 and 90/10, preferentially between 75/25 and 85/15.

Preferably, the legume protein, preferentially pea or faba bean protein, according to the invention has a degree of hydrolysis, or DH, of less than 20, preferentially less than 18, even more preferentially less than 15. The DH will therefore be 0%, 1%, 2%, 3%, 4%, 5%, 6%, 7%, 8%, 9%, 10%, 11%, 12%, 13%, 14%, 15%, 16%, 17%, 18%, 19% or 20%. As well as all the ranges created with these values.

The expression “degree of hydrolysis”, for the purposes of the present invention, means the percentage ratio between the amount of amine (or carboxylic) functions of the free amino acids and the total amount, including the free functions and those engaged in a peptide bond (chemical bond characteristic of proteins resulting from the association of a carboxylic function of a first amino acid and an amine function of a second). For a protein composition consisting of the chain of all its amino acids and thus having only one free amine function and one free carboxylic function, this degree of hydrolysis will be 0%. Conversely, for a protein composition in which the same amino acids are all “free”, that is their two amine and carboxylic functions are not involved in peptide bonds, this degree of hydrolysis will be 100%.

There are several methods for quantifying the degree of hydrolysis. They all consist mainly of the colorimetric assay of the free amine (or carboxylic) functions, followed by the performance of a hydrolysis aimed at destroying all the peptide bonds and finally of a colorimetric assay of the total amine (or carboxylic) functions. The calculated percentage of the free amines (or carboxyls) to the total amines gives the degree of hydrolysis. Any well-known method can be used, such as the TNBS method or the OPA method. In the present invention, the OPA method is preferred, a measurement procedure for which is described hereunder:

The content of amino nitrogen (free NH₂) is determined first of all on the sample of proteins according to the invention with the MEGAZYME kit (reference K-PANOPA). The protein nitrogen content (total nitrogen) of the sample is also determined. It is then possible to calculate the degree of hydrolysis.

### Determining the Content of Amino Nitrogen:

The “amino nitrogen” groups of the free amino acids in the sample react with the N-acetyl-L-cysteine and ophthaldialdehyde (OPA) to form isoindole derivatives.

The amount of isoindole formed during this reaction is stoichiometric with the amount of free amino nitrogen. It is the isoindole derivative which is measured by the increase in absorbance at 340 nm.

A test specimen P*, exactly weighed, of the sample to be analyzed is introduced into a 100 ml beaker. This test specimen will be from 0.5 to 5.0 g based on the amino nitrogen content of the sample. Approximately 50 ml of distilled water is added, homogenization is carried out and the mixture is decanted into a 100-ml graduated flask. 5 ml of 20% sodium dodecyl sulfate (SDS) are added, and the mixture is supplemented with distilled water to reach a volume of 100 ml. Stirring is carried out for 15 minutes with a magnetic stirrer at 1000 rpm. A solution no. 1 is prepared by dissolving a tablet from bottle 1 of the Megazyme kit in 3 ml of distilled water and stirring is carried out until it is completely dissolved. It is necessary to provide one tablet per test. Solution no. 1 is prepared immediately before use.

A blank, a standard and a sample are prepared directly in the spectrophotometer cuvettes under the following conditions:
blank: add 3.00 ml of solution No. 1 and 50 μl of distilled water
standard: add 3.00 ml of solution No. 1 and 50 μl of bottle 3 of the Megazyme kit
sample: introduce 3.00 ml of solution No. 1 and 50 μl of sample preparation.

The content of each cuvette is mixed and the measure of absorbance (A1) of the solutions is taken after about 2 mn in the spectrophotometer at 340 nm (spectrophotometer equipped with cuvettes with 1.0 cm of optical path, able to measure at a wavelength of 340 nm, and verified according to the procedure described in the related manufacturer's technical manual).

The reactions are then immediately initiated by adding 100 μl of solution no. 2, which corresponds to the OPA solution of bottle 2 of the Megazyme kit in each spectrophotometer cuvette.

The content of each cuvette is mixed and they are then placed in darkness for approximately 20 minutes.

The measure of absorbance A2 of the blank, the standard and the sample are then taken from the spectrophotometer at 340 nm.

The free amino nitrogen content, expressed as percentage by weight relative to the weight of the product, is given by the following formula:

$$\% amino nitrogen = \frac{( Δ A e c h - Δ A b l c ) \times 3.15 \times 14.01 \times V \times 100}{6803 \times 0.05 \times m \times 1000}$$

$$\% amino nitrogen = \frac{( Δ A e c h - Δ A b l c ) \times 12.974 \times V}{m \times 1000}$$

where:
ΔAech=Aech2−Aech1
ΔAblc=Ablc2−Ablc1
Aech2=absorbance of sample after addition of solution No. 2
Aech1=absorbance of sample after addition of solution No. 1
Ablc2=absorbance of blank after addition of solution No. 2
Ablc1=absorbance of blank after addition of solution No. 1
V=volume of flask
m=mass of test specimen in g
6803=extinction coefficient of isoindole derivative at 340 nm (in L·mol⁻¹·cm⁻¹).
14.01=molar mass of nitrogen (in g·mol⁻¹)
3.15=final volume in the cuvette (in ml)
0.05=test sample in the cuvette (in ml)

Determining the content of protein nitrogen:

The content of protein nitrogen is determined according to the DUMAS method according to standard ISO 16634-2016. It is expressed as percentage by weight relative to the weight of the product.

### Calculation of the Degree of Hydrolysis

The degree of hydrolysis (DH) is calculated with the following formula:

$$D H = \frac{\% amino nitrogen}{\% protein nitrogen} \times 100$$

According to one embodiment, the legume protein, preferentially pea or faba bean protein, has a solubility at pH 4 and pH 7 ranging from 10% to 99%. The solubility may have all the intermediate amounts (that is, 11%, 12%, 13% . . . 97%, 98%, 99%) and the skilled person will know, on the basis of the method indications given hereinbefore and in the examples section, how to modify the parameters of the method within the indicated ranges in order to achieve the desired solubility. Advantageously, the dried legume protein, preferentially pea or faba bean protein, has a solubility at pH 4 and Ph 7 ranging from 5 to 100%, in particular from 20 to 50% and more preferentially from 20 to 35%. The solubility can be 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%.

### Solubility: Test A

As regards solubility, this is determined using the TEST A method described below:

### Measurement of Solubility in Water

This measurement is based on diluting the sample in distilled water, centrifuging it and analyzing the supernatant.

### Procedure:

Introduce 150 g of distilled water into a 400 ml beaker at 20° C.±2° C., mix with a magnetic stirrer bar and add precisely 5 g of the sample to be tested.

Adjust the pH to the desired value with 0.1 N NaOH or HCl (pH 7), or do not adjust it.

Make up to 200 g with water.

Mix for 30 minutes at 1000 rpm and centrifuge for 15 minutes at 3000 g.

Collect 25 g of supernatant.

Introduce into a previously dried and tared crystallizer.

Place in an oven at 103° C.±2° C. for 1 hour.

Then place in a desiccator (with desiccant) to cool to ambient temperature and weigh.

The soluble dry matter content, expressed in % by weight, is given by the following formula:

$$\frac{( m 1 - m 2 ) \times ( 200 + P ) \times 100}{P 1 \times P} = \% solubility \qquad [ Math . 1 ]$$

Where:
P=weight, in g, of sample=5 g
m1=weight, in g, of crystallizer after drying
m2=weight, in g, of empty crystallizer
P1=weight, in g, of collected sample=25 g

According to one embodiment, the legume protein, preferentially pea or faba bean protein, can have gelling power. This gelling power measured according to test B can range from 1 to 200 Pa, preferentially from 120 Pa to 180 Pa, even more preferentially between 130 Pa and 170 Pa.

### Gelling Power: Test B

The term “gelling power” refers to the functional property which consists of the capacity of a protein composition for forming a gel or a network, which increases the viscosity and generates a state of matter between the liquid and solid states. The term “gel strength” may also be used. To quantify this gelling power, it is thus necessary to generate this network and to evaluate its strength. To carry out this quantification, in the present invention, test B is used, the description of which is as follows:
1) Solubilizing at 60° C.+/−2° C. the protein composition tested in water containing 15%+/−2% solids content at pH 7;
2) Stirring for 5 minutes at 60° C.+/−2° C.;
3) Cooling to 20° C.+/−2° C. and stirring for 24 hours at 350 rpm;
4) Placing the suspension in a controlled-stress rheometer equipped with a concentric cylinder;
5) Measuring elastic modulus G′ and viscous modulus G′ using the following temperature profile:
a. Phase 1: Measuring parameter G′1 after stabilizing at 20° C.+/−2° C. and heating from 20° C.+/−2° C. to 80° C.+/−2° C. in 10 minutes;
b. Phase 2: Stabilizing at a temperature of 80° C.+/−2° C. for 110 minutes;
c. Phase 3: Cooling from 80° C.+/−2° C. to 20° C.+/−2° C. in 30 min and measuring G′2 after stabilization at 20° C.+/−2° C.;
6) Calculating gelling power equal to G′2-G′1.

In a preferred manner, the controlled stress rheometers are chosen from the models DHR 2 (TA, Instruments) and MCR 301 (Anton Paar), with a spindle of concentric cylinder type. They are equipped with a temperature regulation system based on the Peltier effect. In order to avoid evaporation problems at high temperature, liquid paraffin is added on top of the samples.

For the purposes of the invention, a “rheometer” is a laboratory machine for taking measurements regarding the rheology of a fluid or a gel. It applies a force to the sample. Generally of characteristic small dimensions (very small mechanical inertia of the rotor), it allows fundamental study of the mechanical properties of a liquid, a gel, a suspension, a paste, etc., in response to an applied force.

The so-called “controlled stress” models make it possible, by the application of a sinusoidal stress (oscillation mode), to determine the intrinsic viscoelastic values of matter, which notably are dependent upon time (or angular velocity ω) and upon temperature. In particular, this type of rheometer makes it possible to access the complex modulus G*, which itself makes it possible to access the moduli G′ or elastic part and G″ or viscous part;

The first three steps consist in resuspending the protein in water, using precise conditions making it possible to maximize the subsequent measurement.

The chosen water is preferentially reverse osmosis water, but drinking water may also be used.

Its temperature is 60° C.±2° C. during the initial resuspension (1st and 2nd steps) and then 20° C.±2° C. after solubilization for 24 h and cooling before the measurement (3rd step). In general and unless indicated otherwise, when a temperature is given in the present description, it always comprises a variation of ±2° C., for example 20° C.±2° C. or 80° C.±2° C.

A defined amount of protein is added to said water so as to obtain a suspension containing 15%±2% of solids. To do this, equipment such as beakers and stirring bars, well known to those skilled in the art, are used. A volume of 50 ml is stirred for at least 10 h at 350 rpm at room temperature. In general and unless indicated otherwise, the solids contents given in the present description always comprise a variation of ±2%, for example 15%±2%. The pH is adjusted to 7±0.5 using a pH-meter and acid-base reagents, as is well known in the prior art.

The fourth step consists in introducing the sample into the rheometer, and covering said sample with a thin layer of oil in order to limit the evaporation.

During the fifth step, the following temperature protocol is then applied: a. Phase 1: heating from a temperature of 20° C.±2° C. to a temperature of 80° C.±2° C. in 10 minutes; b. Phase 2: stabilization at a temperature of 80° C.±2° C. for 110 minutes; c. Phase 3: cooling from a temperature of 80° C.±2° C. to a temperature of 20° C.±2° C. in 30 min.

The measurement of the parameter G′ is performed continuously during this protocol and is recorded.

The sixth and last step of test B consists in exploiting the recording. Two values are extracted: G′1=value of G′ at the start of phase 1 after stabilization at 20° C.±2° C. and G′2=value of G′ at the end of phase 3 after stabilization at 20° C.±2° C.

The gelling power is equal to G′2−G′1.

According to one optional variant, the legume protein, preferentially pea or faba bean protein, is an enzymatically modified protein. With enzymatically modified protein, the skilled person refers to a protein whose protein structure has been deliberately modified by the addition to the protein of at least one enzyme capable of modifying the protein structure. This enzyme can be selected from proteases, peptidases and deamidation enzymes, for example those of the EC type 3.5.1 such as glutaminase or deamination enzymes, for example those of the EC type 3.5.3 such as peptidylarginine deiminase. These protein-modifying enzymes are known for modifying the physicochemical and/or organoleptic properties of the protein. For example, it is known from document WO2019/233920 A1 that peptidylarginine deiminase reduces the astringency, especially the astringency of rapeseed protein. If the method comprises proteolysis of the protein-enriched fraction, this can modify the degree of hydrolysis (DH) of the protein. Preferably, the degree of hydrolysis is less than 15%, advantageously less than 10%, preferably less than 6%, for example between 3% and 5%. A person skilled in the art will know how to adapt the enzymatic proteolysis conditions, or even will not carry out such a step in order to obtain the desired DH. According to a preferred embodiment of the invention, the legume protein, preferentially pea or faba bean protein, is not enzymatically modified by deamination. According to another preferred variant of the invention, the protein is not enzymatically modified. One advantage of the invention is that it is possible to modify the organoleptic properties of legume protein, preferentially pea or faba bean protein, and especially to give it a milky flavor profile, without even needing to modify the protein enzymatically. According to one embodiment, the invention also makes it possible to provide proteins with an unmodified primary structure.

According to one embodiment, the dried legume protein, preferentially pea or faba bean protein, can have an emulsifying capacity. This emulsifying capacity measured according to test C can range from 60 to 240 ml oil/g, preferentially from 80 to 200 ml oil/g, even more preferentially between 80 and 170 ml oil/g.

To measure ash content, the person skilled in the art will use any method that is well known in the field. Preferably, the person skilled in the art will proceed as follows:
Weigh a sample pea P1
Place the sample 24 h in an oven at 550° C.
Weigh the new sample weight P2
Ash content=(P2/P1)*100.

To measure emulsifying capacity (ml/oil per g), the person skilled in the art will use any method well known in the field. Preferably, the person skilled in the art will proceed as follows according to test C:
1. 0.2 g of the product sample is dispersed in 20 ml of water
2. The solution is homogenized with an Ultraturax IKAT25 for 30 seconds at a speed of 9,500 rpm
3. 20 ml of oil is added under homogenization under the same conditions as step 2 above.
4. Centrifuging is done for 5 minutes at 3100 g
a. If a good emulsion is obtained (no phase shift after 10 seconds), the test is repeated at point 1, increasing the quantities of water and corn oil by 50%.
b. If a poor emulsion is obtained (rapid phase shift), the test is repeated at point 1, reducing the quantities of water and corn oil by 50%.
5. The maximum amount of oil (Qmax in ml) that can be emulsified is thus determined iteratively.
6. The emulsifying capacity is therefore the maximum amount of corn oil that can be emulsified per gram of product.

$$Emulsifying capacity = ( Q max / 0 \text{.2} ) * 100$$

Use of legume protein, preferentially pea or faba bean protein

Another subject matter of the invention is the use of the legume protein, preferentially pea or faba bean protein, of the invention for producing food or beverage products, especially plant-based alternatives to milk.

Generally, the legume protein, preferentially pea or faba bean protein, of the invention can be used in food products and beverages that may include it in an amount up to 100% by weight relative to the total dry weight of the food or beverage product, for example in an amount ranging from about 1% by weight to about 80% by weight relative to the total dry weight of the food or beverage product. All intermediate amounts (that is, 2%, 3%, 4% . . . 77%, 78%, 79% by weight relative to the total weight of the food or beverage product) can be used, as well as all the intermediate ranges based on these quantities. These food and beverage products can be adapted to vegetarian or vegan populations.

A particularly interesting use of the protein of the invention relates to its use in beverages which have a more pleasant taste than those obtained from other commercially available legume proteins. The legume protein, preferentially pea or faba bean protein, of the invention can advantageously be used to produce beverages, in particular milk alternatives, or in other words milk substitutes. Moreover, because of the milky flavor note due to the ingredient, these beverages can also have a more milky flavor note than a beverage not comprising said protein, which is an undeniable advantage for the production of plant-based alternatives to milk. In addition to improving the flavor, the invention also makes it possible to obtain a more velvety mouthfeel than when other legume proteins, preferentially pea or faba bean proteins, are used, which is advantageous for beverages, and especially for plant-based alternatives to milk, as animal milks generally have a velvety mouthfeel.

In beverages, the protein content of these products can vary widely, and can also be a high-protein drink. The protein content can range, for example, from 1% to 12% by dry weight, with respect to the total weight of the beverage, especially from 3% to 10% by dry weight, with respect to the total weight of the beverage. The beverages can be of any type, and include plant-based alternatives to milk or milk substitutes, including barista-type milks and coffee creamers. These may also include other acidic or non-acidic ready-to-drink beverages such as carbonated drinks (including, but not limited to, carbonated soft drinks), non-carbonated drinks (including, but not limited to, non-carbonated soft drinks such as flavored waters, fruit juices and sweetened or unsweetened tea or coffee-based drinks), alcoholic beverages such as beers or spirits, smoothies, and beverage concentrates (including, but not limited to, liquid concentrates and syrups, as well as non-liquid “concentrates” such as freeze-dried and/or powdered preparations or “powder mixes”). It should be noted that in beverages, flavorings or masking agents are generally used to reduce the pea flavor note or bitter aftertaste of the protein, or to flavor the beverage. One of the advantages of the legume protein, preferentially pea or faba bean protein, of the invention is that its use in place of conventional legume proteins, preferentially pea or faba bean proteins, makes it possible to reduce this amount of flavoring or masking agent, or even to totally eliminate these constituents from the beverage altogether, while retaining a very satisfactory taste for the beverage. The beverages can also comprise hydrocolloids; however, since the legume protein, preferentially pea or faba bean protein, provides a more velvety texture, it is possible to reduce or even eliminate the hydrocolloid content while retaining a velvety mouthfeel.

The food products to which it may relate comprise bakery products such as bread products (including, but not limited to, sourdough and unleavened breads, sandwich loaves, yeast breads, and unleavened breads such as baking soda breads), breads containing all types of wheat flour, breads containing all types of flour other than wheat flour (such as potato, rice, barley, spelt, and rye flours), gluten-free breads; mixes for the preparation of said bakery products; sweet bakery products (including, but not limited to, rolls, cakes, pies, pastries, waffles, pancakes, muffins, pancakes, and cookies); mixes for the preparation of said sweet bakery products; pie fillings and other sweet fillings (including, but not limited to, fruit pie fillings and nut pie fillings such as pecan pie fillings, as well as fillings for cookies, cakes, pastries, confectionery products and others, such as cream fillings); and snack bars (including, but not limited to, energy, cereal, nut, and/or fruit bars).

They can also be jellied desserts such as dessert crèmes, custards and puddings. Another type of dessert can also be frozen desserts (including, but not limited to, frozen dairy desserts such as ice cream—including regular ice cream, soft ice cream and all other types of ice cream—and frozen non-dairy desserts such as non-dairy ice cream, sorbet, and others).

Other products conventionally prepared using animal milk can also comprise the legume protein, preferentially pea or faba bean protein, of the invention to form substitutes. These products may be acidified and/or fermented with ferments, such as lactic, vegan, or mesophilic ferments. These may include yoghurts (including, but not limited to, full-fat, low-fat, and fat-free yoghurts, which may be milk protein-free and lactose-free). The term “yoghurt” also includes fromage frais and petits-suisses. It may also include cheese substitutes such as cheese spreads, melted cheeses, pressed cooked and uncooked cheeses, soft cheeses, spun cheeses, blue cheeses; it may include Emmental, string cheese, ricotta, provolone, Parmesan, Munster, mozzarella, Monterey Jack, Manchego, blue, fontina, feta, Edam, double Gloucester, Camembert, Cheddar, Brie, Asiago and havarti. It can also include other products such as plant-based butters and even crème fraiche.

Other products that can include the legume protein, preferentially pea or faba bean protein, of the invention are sauces such as vinaigrettes or mayonnaise- or ketchup-based sauces or syrups.

Likewise, the legume proteins, preferentially pea or faba bean proteins, of the invention can be incorporated into confectionery products (including, but not limited to, jelly candies, soft candies, hard candies, chocolates, caramels, and gums); sweetened and unsweetened breakfast cereals (including, but not limited to, extruded cereals, flaked cereals, and puffed cereals); and cereal coating compositions for the preparation of breakfast cereals. These may also include sweet spreads (including, but not limited to, jellies, jams, nut butters such as peanut butter, spreads, and other spreadable products).

The legume proteins, preferentially pea or faba bean proteins, of the invention can also be used as flavor carriers or encapsulants.

Other types of food and beverages that are not mentioned herein but which conventionally comprise one or more proteins can also be envisaged in the context of the present invention. In particular, animal feed (such as pet food) is explicitly envisaged.

Legume protein, preferentially pea or faba bean protein, can also be used, optionally after texturing, in meat substitutes such as emulsified sausages or hamburgers, or in fish or seafood substitutes. It can also be used in egg-replacement formulations or in the production of protein products such as tofu or tempeh. Textured proteins generally refers to proteins textured by extrusion, i.e. dry extrusion or textured vegetable protein, or high moisture extrusion. The extruders can be single-screw, twin-screw or multi-screw. In the case of twin-screw extrusion, the extrusion can be co-rotating or counter-rotating. As examples of multi-screw extrusion, mention may be made of the planetary extruder and the ring extruder. Other more specific technologies can include shear cell technology, microextrusion and 3D printing.

Food products or beverages can be used especially in specialized nutrition, for example for specific populations, such as babies or infants, children, adolescents, adults, the elderly, athletes, people suffering from a disease, etc. These may be meal-replacement formulas or complete nutritional beverages, for example for weight management or clinical nutrition (e.g. tube feeding or enteral nutrition).

The legume protein can be used as a single source of proteins, but can also be used in combination with other additional plant or animal proteins. These additional proteins may be hydrolyzed or non-hydrolyzed. These additional proteins are generally in the form of concentrates or isolates. A distinction is made between concentrates and isolates according to their protein content: concentrates with a protein content of between 50% and 70% and isolates with a protein content of over 70%, preferentially between 80% and 90%, respectively. The term “plant protein” denotes all the proteins derived from cereals, oleaginous plants, leguminous plants and tuberous plants, as well as all the proteins derived from algae and microalgae or fungi, used alone or as a mixture, selected from the same family or from different families. “Leguminous plants” generally refers, in the present application, to the family of dicotyledonous plants of the Fabales order. Several leguminous plants are significant crop plants, such as soybean, beans, especially the mung bean, chickpea, faba bean, groundnut, cultivated lentil, cultivated alfalfa, various clovers, broad beans, locust bean, licorice, and lupin. The additional legume protein can be selected from these legumes or else can be a legume protein such as that used in the invention. In the present application, the term “cereals” refers to plants cultivated from the family of grasses producing edible grains, for example wheat, oat, rye, barley, corn, sorghum, or rice. Tubers may be carrot, cassava, konjac, potato, Jerusalem artichoke, sweet potato. Oilseed plants are generally plants that produce seeds from which oil is extracted. Oilseed plants can be selected from sunflower, rapeseed, peanut, sesame, squash, or flax. The animal proteins can, for example, be egg or milk proteins, such as whey proteins, casein, or caseinate proteins. The legume protein composition, preferentially of the invention can thus be used in association with one or more of these proteins or amino acids in order to improve the nutritional properties of the end product, for example to improve the PDCAAS of the protein or to provide other functionalities.

Legume protein, preferentially pea or faba bean protein, can also be used for producing pharmaceuticals or in fermentation, for example for producing fungal metabolites or cell culture metabolites.

The invention and its advantages will now be illustrated in the embodiments detailed in the examples section hereunder. It should be noted that these examples do not limit the present invention.

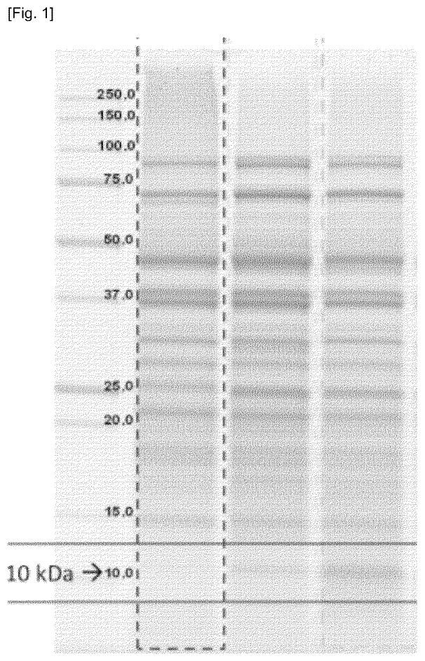

FIG. 1

FIG. 1 shows an electrophoretic profile. The left-hand column of the photo shows several bands whose values, expressed in kilodaltons (kDa), are 10.0; 15.0; 20.0; 25.0; 37.0; 50.0; 75.0; 100.0; 150.0 and 250.0.

The electrophoretic profile is performed by migration of the sample under denaturing and reducing conditions through a 20% polyacrylamide gel followed by Coomassie Blue staining.

### EXAMPLES

### Example 1: Isolate Obtained According to the Invention

About 1000 kg of peas were used. The external fibers of the peas were first separated from the seeds by crushing (mechanically separating the external hull and the pea seeds) and skinning (sorting the external hulls and the skinned seeds using compressed air). Water at 75° C. was used in this first part of the method: pea seeds and water were fed continuously into a Bruynooghe immersed-screw blancher. The water/pea weight ratio was approximately 1. All of the water entered at the entrance to the blancher and the peas were immediately introduced into the water at 75° C. As soon as the peas were in contact with the water, the temperature of the suspension was about 55° C. The screw speed was set so that the peas passed through the blancher in 3 minutes, and the temperature remained stable during this period. Water was added at the outlet of the blancher to give a water/pea weight ratio of about 1.5. The suspension was immediately milled by continuous wet milling, introducing water at room temperature during milling, to obtain a milled pea seed suspension with about 20% solids content at a temperature of about 35° C.

The milled pea seed suspension was adjusted to pH 8.5 by continuously adding sodium hydroxide online. The milled pea seed suspension was cooled to about 10° C. by passing through a plate heat exchanger and then transferred to a stirred storage tank. This milled pea seed suspension was fed into a centrifugal decanter (Flottweg Z3). The protein-enriched fraction was recovered from the overflow (about 7% solids content).

The protein-enriched fraction then undergoes ultrafiltration on a SYNDER MAX ST-4B-2540M membrane with a 10 KDa cut-off. Transmembrane pressure is about 3.5 bar. The filtration temperature is 45° C. The ultrafiltration retentate is diafiltered at constant volume with 3 volumes of water in order to reduce the concentration of impurities and obtain a protein retentate with a protein content of at least 70%.

The protein retentate was adjusted to pH 7.0 using sodium hydroxide and hydrochloric acid, then heat-treated at 130° C. for 5 seconds and flash-cooled to about 75° C. The protein-enriched fraction was lastly passed through a 200-bar high-pressure homogenizer and then atomized in a TGE nozzle atomizer. The pea protein powder named “Pea isolate according to example 1” was then analyzed.

### Example 2: Influence of the Pea Seed Preparation Step

The pea seed preparation step in paragraph 122 of Example 1 is varied in order to compare the impact of the following two variants:
Dry-milled pea seed, without heat treatment
the seed is therefore simply milled dry
then set to 20% dry matter and pH 8.5
the suspension is fed into a centrifugal decanter (Flottweg Z3) to recover the overflow
A soaked pea seed
The pea seed is placed in demineralized water heated to 20° C. for 16 hours.
The seed is then milled wet
then set to 20% dry matter and pH 8.5
the suspension is fed into a centrifugal decanter (Flottweg Z3) to recover the overflow

The extraction method is then similar in every respect to that described in paragraphs 124 to 125.

The result is a “Pea protein isolate according to example 2” and a “Pea protein isolate according to example 3” for the methods using dry-milled and soaked-milled pea seeds, respectively.

It should first be noted that the ultrafiltration carried out to produce the “Pea isolate according to example 2” (that is, obtained from pre-soaked pea seeds) was excessively complicated to produce, as the flow rate dropped rapidly, certainly as a result of soaking. In fact, such soaking causes natural fermentation, resulting in the dissolution of numerous compounds, including polysaccharides. These have a high clogging capacity, which explains the difficulty in filtering.

Table 1 below compares the compositions of the different protein compositions obtained.

TABLE 1

| | Pea isolate | | | |
|---|---|---|---|---|
| | according | | | |
| | to | Pea isolate | | |
| | example 1 | according | Pea isolate | |
| | blanched | to | according | |
| | and milled - | example 2 | to | |
| | according to | dry | example 2 | NUTRALYS ® |
| | the invention | milling | soaked | F85M |
| Proteins | 75.8% | 76.2% | 75.7% | 85.10% |
| Carbohydrates | 12.3% | 12.0% | 12.1% | 0.1% |
| of which, | 0.1 | 0.5 | 0.2 | Nd |
| glucose | | | | |
| of which, | <0.1 | 0.5 | 0.1 | Nd |
| fructose | | | | |
| of which, | 0.9 | 0.7 | 0.6 | Nd |
| sucrose | | | | |
| of which, | <0.1 | <0.1 | <0.1 | Nd |
| melibiosis | | | | |
| of which, | 0.3 | 0.9 | 0.3 | Nd |
| raffinose | | | | |
| of which, | 2.9 | 3.7 | 2.9 | Nd |
| stachyose | | | | |
| of which, | 1.7 | 2.3 | 1.9 | Nd |
| verbascose | | | | |
| Lipids | 6.5% | 6.4% | 6.5% | 8.4% |
| Ash | 5.4% | 5.4% | 5.7% | 4.3% |
| Solubility at | 27.8% | 32.7% | 28.2% | 60% |
| pH 4 according | | | | |
| to test A | | | | |
| Solubility at | 31.0% | 76.7% | 39.5% | 15% |
| pH 7 according | | | | |
| to test A | | | | |
| Gel strength | 144 | 17 | 110 | 125 |
| according to | | | | |
| test B | | | | |
| Emulsifying | 100 | 250 | 50 | 500 |
| capacity (ml | | | | |
| oil per g) | | | | |
| according to | | | | |
| test C | | | | |

Nd means not detected.

An organoleptic analysis of the different compositions is also carried out. The method used is called “Block Profiling” and is analyzed using FIZZ software. 12 panelists take part in the panel. Samples are resuspended at 4% in Evian water and homogenized. Samples are coded using a 3-digit code, presented in random order in a white-lit cubicle. The descriptors analyzed are: chalky, salty, bitter, astringent, pea, potato, broth, nut.

Table 2 summarizes the results obtained:

TABLE 2

| | Pea protein | Pea protein | Pea protein |
|---|---|---|---|
| | isolate | isolate | isolate |
| | according to | according to | according to |
| | example 1 | example 2 | example 3 |
| | (blanched) | (dry milled) | (soaked) |
| Chalky | 2.1 | 2.0 | 6.6 |
| Salty | 2.7 | 4.0 | 3.5 |
| Bitter | 3.5 | 5.0 | 6.0 |
| Astringent | 3.2 | 5.5 | 6.0 |
| Pea | 3.1 | 3.1 | 3.0 |
| Potato | 3.0 | 2.5 | 1.4 |
| Broth | 2.0 | 2.0 | 3.0 |
| Nut | 1.5 | 1.5 | 0.8 |

In short, the combination of the pea seed bleaching step and ultrafiltration enables a pea protein to be obtained productively whose gel strength and emulsifying power are on a par with the values of a pea protein isolate conventionally obtained by isoelectric precipitation (e.g. NUTRALYS® F85M).

The combination of soaking and ultrafiltration is complicated from a filtration rate standpoint, and results in an isolate with an undesirable organoleptic profile (particularly chalky, astringent and bitter).

The combination of dry milling and ultrafiltration does not present these problems, but the gel strength is very low compared with a conventional isolate.

### Example 3: Influence of Ultrafiltration Cutoff Selection

In this example, Example 1 is reproduced with ultrafiltration membranes having a cut-off of 5 KDa, 10 KDa and 50 KDa (recommended by all the prior art, including the Fuhrmeister article). The membranes used belong to the SYNDER MAX ST-4B-2540M range, and each has the cutoff threshold advertised.

Electrophoresis is performed on the different retentates obtained. The result is shown in FIG. 1.

The framed 10 KDa band consists mainly of albumin. Nutralys® (left track) is essentially devoid of them, which is consistent with its isoelectric precipitation method. Clearly, the product according to the invention (right track) has a denser band than the prior art (middle track, cut-off 50 KDa). The compositions of the product according to the invention and the product according to the recommendation of the prior art (in particular the Fuhrmeister article) are therefore at least different in terms of albumin content.

1. A method for producing legume protein, preferentially pea or faba bean protein, comprising the following steps:
1. Preparation of an aqueous suspension of legume seeds, preferentially pea or faba bean seeds, milled in an aqueous solution, said preparation being carried out in the presence of a heat treatment;
2. Removal of an insoluble fraction by solid liquid separation of the aqueous suspension of milled legume seeds, preferentially pea or faba bean seeds, prepared in step 1) to obtain a protein-enriched fraction;
3. Ultrafiltration of the protein-enriched fraction and removal of a permeate to generate a retentate.

2. The method according to claim 1, wherein the heat pre-treatment in step 1 comprises:
a) introducing legume seeds, preferentially pea or faba bean seeds, or milled legume seeds, preferentially pea or faba bean seeds, into an aqueous solution at a temperature of between 65° C. and 90° C. so as to obtain an aqueous composition;
b) heat treating the aqueous composition obtained in step a) formed between the legume seeds, preferentially pea or faba bean seeds, or the milled legume seeds, preferentially pea or faba bean seeds, and the aqueous solution at a temperature of between 40° C. and 65° C. for 1 to 10 min.

3. The method according to claim 1, wherein the ultrafiltration in step 3) is carried out using a membrane with a cut-off value of between 5 KDa and 10 KDa.

4. The method according to claim 1, wherein the ultrafiltration step comprises a diafiltration carried out to achieve a minimum protein content of 70% in the retentate.

5. A legume protein composition, preferentially pea or faba bean protein composition, that can be obtained according to the method according to claim 1.

6. The legume protein composition, preferentially pea or faba bean protein composition, according of claim 5, wherein the legume protein composition comprises a protein content of between 70% and 90% expressed in grams of protein per 100 g of dry matter, said proteins consisting of a mixture of globulins and albumins.

7. The legume protein composition, preferentially pea or faba bean protein composition, according to claim 5, wherein the dry weight ratio of globulins to albumins is between 70/30 and 90/10, preferentially between 75/25 and 85/15.

8. The legume protein composition, preferentially pea or faba bean protein composition, according to claim 5, wherein the legume protein composition has a gelling power according to test B between 1 and 200 Pa, preferentially from 120 Pa to 180 Pa, even more preferentially between 130 Pa and 170 Pa.

9. A use of the legume protein, preferentially pea or faba bean protein, that can be obtained according to claim 1 for producing food or beverage products, especially plant-based alternatives to milk.

10. The method according to claim 2, wherein the ultrafiltration in step 3) is carried out using a membrane with a cut-off value of between 5 KDa and 10 KDa.

11. The legume protein composition, preferentially pea or faba bean protein composition, according to claim 6, wherein the dry weight ratio of globulins to albumins is between 70/30 and 90/10, preferentially between 75/25 and 85/15.
